(19) United States
(12) Patent Application Publication
(10) Pub. No.: US 2019/0355174 A1
(43) Pub. Date: Nov. 21, 2019

(54) INFORMATION PROCESSING APPARATUS, INFORMATION PROCESSING SYSTEM, INFORMATION PROCESSING METHOD, AND COMPUTER-READABLE RECORDING MEDIUM

(71) Applicant: CANON KABUSHIKI KAISHA, Tokyo (JP)

(72) Inventors: Takaaki Endo, Urayasu-shi (JP); Kiyohide Satoh, Kawasaki-shi (JP)

(21) Appl. No.: 16/530,772

(22) Filed: Aug. 2, 2019

Related U.S. Application Data
(63) Continuation of application No. 13/516,661, filed on Jul. 3, 2012.

(30) Foreign Application Priority Data
Dec. 18, 2009 (JP) 2009-288454

(51) Int. Cl. G06T 19/00; A61B 6/00; A61B 8/00; A61B 8/08

(52) U.S. Cl. CPC G06T 19/00; A61B 6/5247; A61B 8/565; A61B 8/5261; A61B 5/055; A61B 8/523; A61B 8/483; A61B 8/5223; A61B 6/00; A61B 8/5246

(57) ABSTRACT

An information processing apparatus includes a display control unit configured to control display of a cross-sectional image along a first cross section passing through a subject and a cross-sectional image along a second cross section passing through a specified position of the subject; an acquisition unit configured to acquire an inclination of the first cross section with respect to the subject; and a setting unit configured to set the second cross section as a cross section that is parallel to the first cross section and that passes through the specified position on the basis of the acquired inclination.

### TECHNICAL FIELD

The present invention relates to an information processing apparatus, an information processing system, an information processing method, and a computer-readable recording medium for displaying multiple images.

### BACKGROUND ART

Image diagnosis using medical images is in widespread use in medical fields. In the image diagnosis, medical images captured by imaging apparatuses are displayed on monitors and doctors read the displayed images to diagnose lesion areas. Among the medical images, tomographic images resulting from imaging of inner parts of subjects are particularly useful for the diagnosis. Medical image acquisition apparatuses (hereinafter referred to as modalities) capturing tomographic images include ultrasonic diagnostic imaging apparatuses, magnetic resonance imaging apparatuses (hereinafter referred to as MRI apparatuses), and X-ray computed tomographic apparatuses (hereinafter referred to as X-ray CT apparatuses).

Comparison between multiple tomographic images captured by multiple modalities and comparison between lesion areas in tomographic images captured at different dates and times are performed nowadays. The comparison is intended to more accurately diagnose the states of the lesion areas.

In order to use multiple tomographic images of the same subject for the diagnosis, it is necessary to perform registration to associate the tomographic images with each other. In the manual registration approach, operators such as doctors manually perform the registration while watching the images with the object of giving importance to the accuracy. It is necessary for the operators to find the corresponding positions from the multiple tomographic images on the basis of the similarity in the shapes of the lesion areas, the appearance of their peripheral parts, or the like.

A technology, to display an ultrasonic tomographic image and an image of a cross section that is captured by an X-ray CT apparatus, is adopted in order to aid the manual registration. In this technology, the ultrasonic tomographic image is displayed in response to an operation with an ultrasound probe, while the X-ray image is displayed in a still state and includes a target lesion area. In this case, the user operates the ultrasonic imaging apparatus to search for an ultrasonic tomographic image including a corresponding lesion area while comparing the ultrasonic tomographic image with the X-ray still image. A technology to constantly display a cross-sectional image including a lesion area and rotate a target cross section in an arbitrary direction is also adopted.

With the above technologies, the user is required to perform the registration between the target lesion area and the corresponding lesion area and required to match the inclinations of the cross-sectional images with each other. These operations impose heavy burden on the user and it takes longer time to carry out the operations.

Accordingly, a technology is needed to provide display for aiding the accurate registration while relieving the burden on the operator.

### SUMMARY OF INVENTION

According to an embodiment of the present invention, an information processing apparatus includes a display control unit configured to control display of a cross-sectional image along a first cross section passing through a subject and a cross-sectional image along a second cross section passing through a specified position of the subject; an acquisition unit configured to acquire an inclination of the first cross section with respect to the subject; and a setting unit configured to set the second cross section as a cross section that is parallel to the first cross section and that passes through the specified position on the basis of the acquired inclination.

Further features of the present invention will become apparent from the following description of exemplary embodiments with reference to the attached drawings.

### BRIEF DESCRIPTION OF DRAWINGS

FIG. 1 is a block diagram showing an example of the configuration of an information processing system according to a first embodiment of the present invention.

FIG. 2 is a block diagram showing an example of the basic configuration of a computer capable of realizing the blocks in an information processing apparatus according to the first embodiment by software.

FIG. 3 shows an outline of how to generate a cross-sectional image corresponding to an ultrasonic tomographic image from MRI volume data.

FIG. 4 is a flowchart showing an example of the overall process performed by the information processing apparatus according to the first embodiment.

FIG. 5A illustrates an example of how to combine and display cross-sectional images.

FIG. 5B illustrates another example of how to combine and display cross-sectional images.

FIG. 6 is a block diagram showing an example of the configuration of an information processing system according to a second embodiment of the present invention.

FIG. 7 is a flowchart showing an example of the overall process performed by an information processing apparatus according to the second embodiment.

FIG. 8 is a flowchart showing an example of a process of selecting a tomographic image.

### DESCRIPTION OF EMBODIMENTS

### First Embodiment

An information processing system according to a first embodiment of the present invention extracts a cross-sectional image that has the same orientation as that of an ultrasonic tomographic image being captured in real time and that includes a target lesion area from three-dimensional image data. This allows an operator (a doctor or an engineer) to easily find a tomographic image (a cross-sectional image) that includes an area (a corresponding lesion area) in the three-dimensional image data corresponding to the target lesion area.

An image along an arbitrary cross section in a three-dimensional image is hereinafter referred to as a cross-sectional image. The cross-sectional image is referred to as a tomographic image when the fact that the cross-sectional image is captured by a tomographic imaging apparatus using ultrasonic waves or the likes is emphasized. A case in which a tomographic image group representing three-dimensional information inside a subject is processed as the three-dimensional image data will now be described here.

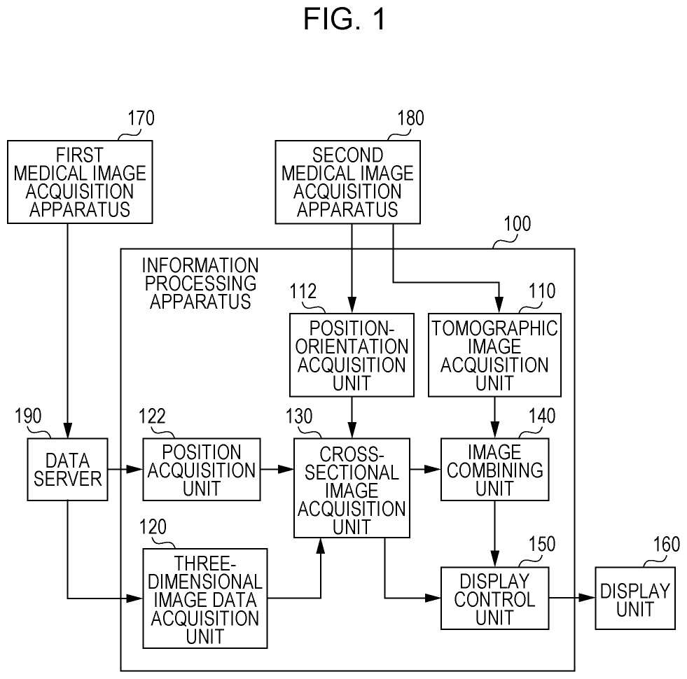

FIG. 1 is a block diagram showing an example of the configuration of the information processing system according to the first embodiment of the present invention. Referring to FIG. 1, an information processing apparatus 100 includes a tomographic image acquisition unit 110, a position-orientation acquisition unit 112, a three-dimensional image data acquisition unit 120, a position acquisition unit 122, a cross-sectional image acquisition unit 130, an image combining unit 140, and a display control unit 150. The information processing apparatus 100 is connected to a data server 190 holding three-dimensional image data and a second medical image acquisition apparatus 180 capturing an ultrasonic tomographic image of a subject.

The data server 190 holds a reference tomographic image group of the subject captured by, for example, an MRI apparatus or an X-ray CT apparatus serving as a first medical image acquisition apparatus 170. A case in which the MRI apparatus is used as the first medical image acquisition apparatus 170 is exemplified here.

The position and orientation of each tomographic image composing the reference tomographic image group is represented in an MRI apparatus coordinate system. The MRI apparatus coordinate system means a coordinate system defined by using one point in a space based on the MRI apparatus as the origin. The three-dimensional image data represented in the MRI apparatus coordinate system is supplied to the information processing apparatus 100 through the three-dimensional image data acquisition unit 120.

The data server 190 also holds the position of a lesion area (a target lesion area) that is specified in advance as a target area in the three-dimensional image data. The position of the target lesion area is specified by the operator who selects a tomographic image including the target lesion area from the reference tomographic image group on an image viewer (not shown) and clicks the target lesion area with a mouse (not shown). The position of the target lesion area held by the data server 190 is supplied to the information processing apparatus 100 through the position acquisition unit 122. The position of the target lesion area is also represented in the MRI apparatus coordinate system, like the three-dimensional image data, in the following description.

The ultrasonic diagnostic imaging apparatus serving as the second medical image acquisition apparatus 180 captures an ultrasonic tomographic image of the subject in real time. The ultrasonic tomographic images captured by the ultrasonic diagnostic imaging apparatus are sequentially supplied to the information processing apparatus 100 through the tomographic image acquisition unit 110.

The operator normally captures an image of the subject while moving an ultrasound probe, which is an image capturing unit in the ultrasonic diagnostic imaging apparatus and which carried with a hand of the operator. Accordingly, it is not apparent that the ultrasonic tomographic image corresponds to which position and orientation in a space based on the subject. According to the first embodiment of the present invention, a position-orientation sensor (not shown) is mounted in the ultrasonic diagnostic imaging apparatus to measure the position and orientation of the ultrasound probe. For example, FASTRACk manufactured by Polhemus in U.S. is used as the position-orientation sensor. The position-orientation sensor may have any structure as long as it is capable of measuring the position and orientation of an ultrasound probe.

The position and orientation of the ultrasound probe measured in the above manner is supplied to the information processing apparatus 100 through the position-orientation acquisition unit 112. The position and orientation of the ultrasound probe is represented in, for example, a reference coordinate system. The reference coordinate system means a coordinate system defined by using one point in a space based on the subject as the origin. It is assumed here that the positions and orientations of the ultrasound probe and various images are defined in the reference coordinate system, unless otherwise specified. The position and orientation of the ultrasound probe may be input in advance by the operator with a keyboard or mouse (not shown). The position and orientation of the ultrasound probe is used to define a first cross section passing through the subject to generate a two-dimensional cross-sectional image of the subject included in the first cross section.

The tomographic image acquisition unit 110 acquires the ultrasonic tomographic image supplied to the information processing apparatus 100 as a first two-dimensional cross-sectional image. The tomographic image acquisition unit 110 converts the ultrasonic tomographic image into digital data, if needed, and associates the digital data with the position and orientation acquired by the position-orientation acquisition unit 112. The tomographic image acquisition unit 110 supplies the ultrasonic tomographic image to the image combining unit 140.

The position-orientation acquisition unit 112 calculates the position and orientation of the ultrasonic tomographic image or the inclination of the cross section including the ultrasonic tomographic image with respect to the subject on the basis of the position and orientation of the ultrasound probe. The position-orientation acquisition unit 112 associates the position and orientation of the ultrasonic tomographic image or the inclination with the ultrasonic tomographic image acquired by the tomographic image acquisition unit 110 to hold the position and orientation of the ultrasonic tomographic image or the inclination associated with the ultrasonic tomographic image acquired by the tomographic image acquisition unit 110. The position-orientation acquisition unit 112 supplies the held position and orientation to the cross-sectional image acquisition unit 130 in response to a request from the cross-sectional image acquisition unit 130. The position-orientation acquisition unit 112 acquires the position of the corresponding lesion area specified by the operator to correct the position of the ultrasonic tomographic image by the amount of offset from the position of the target lesion area.

The three-dimensional image data acquisition unit 120 acquires the three-dimensional image data (the reference tomographic image group) supplied to the information processing apparatus 100 to hold the three-dimensional image data (the reference tomographic image group). The three-dimensional image data acquisition unit 120 supplies the held three-dimensional image data to the cross-sectional image acquisition unit 130 in response to a request from the cross-sectional image acquisition unit 130.

The position acquisition unit 122 acquires the position of the target lesion area supplied to the information processing apparatus 100 to hold the position of the target lesion area. The position acquisition unit 122 supplies the held position of the target lesion area to the cross-sectional image acquisition unit 130 in response to a request from the cross-sectional image acquisition unit 130.

The cross-sectional image acquisition unit 130 receives the three-dimensional image data from the three-dimensional image data acquisition unit 120 and the position of the target lesion area from the position acquisition unit 122. The cross-sectional image acquisition unit 130 also receives the position and orientation of the ultrasonic tomographic image from the position-orientation acquisition unit 112. The cross-sectional image acquisition unit 130 generates a cross-sectional image (a second two-dimensional cross-sectional image) that has the same orientation (the same inclination with respect to the subject) as that of the ultrasonic tomographic image and that includes the target lesion area on the basis of the above data.

The image combining unit 140 receives the ultrasonic tomographic image from the tomographic image acquisition unit 110 and the cross-sectional image from the cross-sectional image acquisition unit 130. The image combining unit 140 combines the received ultrasonic tomographic image with the received cross-sectional image to generate a combined image and supplies the combined image to the display control unit 150 or an external apparatus.

The display control unit 150 acquires the combined image from the image combining unit 140 and displays the acquired combined image in the display unit 160. The operator can compare the two cross-sectional images in the combined image with each other to determine whether the image captured by the ultrasound probe includes the lesion area, which is the target area. If the same lesion area is included in the two cross-sectional images, it is determined that the lesion area exists at the position of the subject where the ultrasound probe is pressed. In addition, this results in the registration between the ultrasonic tomographic image or the subject and the three-dimensional image data from the MRI apparatus (the MRI three-dimensional image data).

Part or all of the blocks shown in FIG. 1 may be provided as independent apparatuses. Alternatively, the blocks are installed in one or more computers and are executed by the central processing units (CPUs) in the computers to realize the blocks as software realizing the functions of the blocks. It is assumed in the first embodiment that the blocks are realized by software and are installed in the same computer.

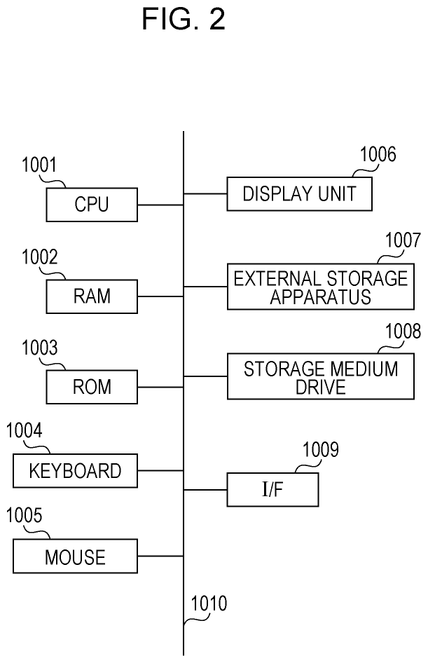

FIG. 2 is a block diagram showing an example of the basic configuration of hardware for realizing the functions of the information processing apparatus 100 shown in FIG. 1 by the software.

Referring to FIG. 2, a CPU 1001 uses programs and data stored in a random access memory (RAM) 1002 or a read only memory (ROM) 1003 to control the entire computer. The CPU 1001 controls execution of the software corresponding to the respective blocks in FIG. 1 to realize the functions of the components.

The RAM 1002 includes an area in which the loaded programs and data are temporarily stored and a working area necessary for the CPU 1001 to perform a variety of processing.

The ROM 1003 generally stores the programs and setup data of the computer. A keyboard 1004 and a mouse 1005 are input devices and the operator uses the keyboard 1004 and the mouse 1005 to input various instructions into the CPU 1001.

A display unit 1006 is, for example, a cathode ray tube (CRT) or a liquid crystal display and corresponds to the display unit 160 in FIG. 1. The display unit 1006 displays, for example, a message and/or a graphical user interface (GUI) to be displayed for image processing, in addition to the combined image generated by the image combining unit 140.

An external storage apparatus 1007 is, for example, a hard disk drive that stores programs executed by an operating system (OS) and the CPU 1001. The information described in the first embodiment is stored in the external storage apparatus 1007 and is loaded in the RAM 1002, if needed.

A storage medium drive 1008 reads out a program or data stored in a storage medium, such as a compact disc-read only memory (CD-ROM) or a digital versatile disk-read only memory (DVD-ROM), in response to an instruction from the CPU 1001.

An interface (I/F) 1009 includes, for example, a digital input-output port conforming to Institute of Electrical and Electronics Engineers (IEEE) 1394 or the like and an Ethernet port through which information including the combined image is externally output. The data input through the digital input-output port and the Ethernet port is supplied to the RAM 1002 through the I/F 1009. Part of the functions of the tomographic image acquisition unit 110, the position-orientation acquisition unit 112, the three-dimensional image data acquisition unit 120, and the position acquisition unit 122 is realized by the I/F 1009.

The components described above are connected to each other via a bus 1010.

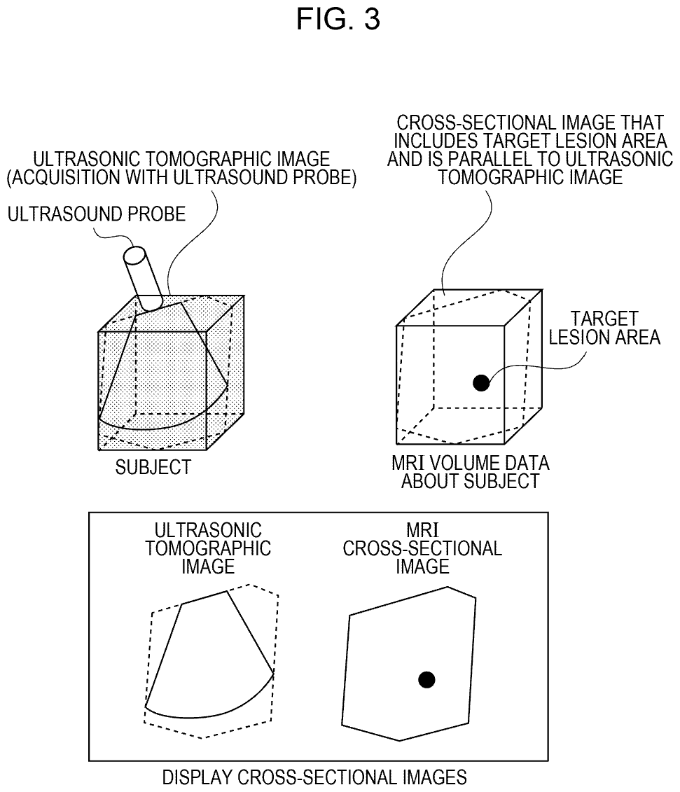

An outline of processing realized by the above information processing system will now be described with reference to FIG. 3. The processing causes the display unit 160 to display an ultrasonic tomographic image and a cross-sectional image generated (acquired) from three-dimensional image data from the MRI apparatus in association with the ultrasonic tomographic image. This is intended to perform the registration between the MRI three-dimensional image data and the ultrasonic tomographic image. A subject and an ultrasound probe are shown in an upper left part in FIG. 3. Volume data generated from the MRI three-dimensional image data and a cross-sectional image generated on the basis of an ultrasonic tomographic image are shown in an upper right part in FIG. 3. How the ultrasonic tomographic image acquired by the ultrasound probe and the MRI cross-sectional image generated from the MRI volume data are displayed is shown in a lower part in FIG. 3.

The operator (for example, doctor or engineer) presses the ultrasound probe on the subject to acquire the ultrasonic tomographic image of the subject. In the upper left part in FIG. 3, an ultrasonic tomographic image is represented by a solid line and a plane including the ultrasonic tomographic image is represented by a broken line. Since the position and orientation of the ultrasound probe can be measured with the sensor, information about the position and orientation of the ultrasonic tomographic image with respect to the subject can be acquired.

On the MRI three-dimensional image data, the lesion area is manually identified by the operator or is identified by the image processing. The target to be identified is not limited to the lesion area and may be any target area where a feature shape or the like appears. The operator searches the ultrasonic tomographic image of the subject for the area identified on the MRI three-dimensional image data.

The information processing system described above generates (acquires) the cross-sectional image from the MRI three-dimensional image data on the basis of the position and orientation of the ultrasonic tomographic image and the position of the target area (target lesion area). The cross-sectional image that is generated here is parallel to the cross section including the ultrasonic tomographic image and passes through the target area. The inclinations of the two cross-sectional images (the ultrasonic tomographic image and the MRI cross-sectional image) with respect to the subject can be constantly matched with each other for display in the above manner, regardless of the orientation of the ultrasound probe. As a result, the operator can match the positions and orientations of the two cross-sectional images with each other by appropriately matching only the position where the ultrasound probe is pressed with the data from the MRI apparatus. Consequently, it is possible to save the trouble to match the inclinations, thus facilitating the registration by the operator.

The ultrasonic tomographic image and the MRI cross-sectional image are displayed in the display unit 160. The operator performs the registration by comparing the content of the ultrasonic tomographic image with the content of the MRI cross-sectional image while varying the position where the ultrasound probe is pressed.

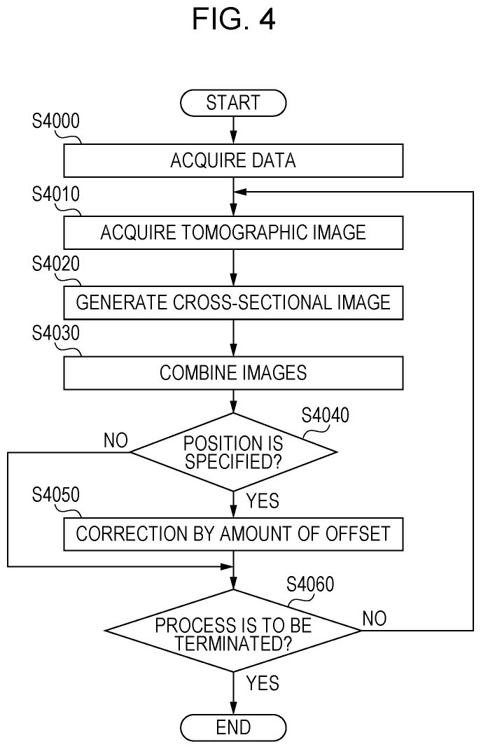

FIG. 4 is a flowchart showing an example of the overall process performed by the information processing apparatus 100. The steps in the flowchart in FIG. 4 are realized by the CPU 1001 that executes the programs realizing the functions of the respective components. It is assumed that, before the following process is started, the program code in accordance with the flowchart has been loaded in the RAM 1002 from, for example, the external storage apparatus 1007.

### (S4000) Acquisition of Three-Dimensional Image Data

In Step S4000, the three-dimensional image data acquisition unit 120 acquires a reference tomographic image group from the data server 190 as three-dimensional image data. The position acquisition unit 122 acquires the position of the target area (the target lesion area) from the data server 190. The three-dimensional image data acquisition unit 120 converts the coordinate system of the reference tomographic image group data from the MRI apparatus coordinate system into the reference coordinate system.

### (S4010) Acquisition of Tomographic Image

In Step S4010, the tomographic image acquisition unit 110 in the information processing apparatus 100 acquires an ultrasonic tomographic image from the second medical image acquisition apparatus 180. The position-orientation acquisition unit 112 in the information processing apparatus 100 acquires the position and orientation of the ultrasound probe at the time when the ultrasonic tomographic image is captured from the second medical image acquisition apparatus 180. The information processing apparatus 100 calculates the position and orientation of the ultrasonic tomographic image from the position and orientation of the ultrasound probe by using the relative relationship between the positions and orientations of the ultrasound probe and the ultrasonic tomographic image that are stored.

### (S4020) Generation of Cross-Sectional Image

In Step S4020, the cross-sectional image acquisition unit 130 generates a cross-sectional image from the three-dimensional image data on the basis of the position of the target lesion area and the position and orientation of the ultrasonic tomographic image.

First, the cross-sectional image acquisition unit 130 restores three-dimensional volume data in which the luminance value of each three-dimensional voxel is stored from the reference tomographic image group acquired in Step S4000 as pre-processing. This processing is performed by three-dimensional arrangement and interpolation of each pixel in each tomographic image. It is sufficient to perform the pre-processing only once when Step S4020 is first executed.

Then, the cross-sectional image acquisition unit 130 calculates a cross section (plane) based on the position of the target lesion area and the orientation of the ultrasonic tomographic image. Specifically, first, the cross-sectional image acquisition unit 130 initializes the position and orientation of the cross section in a cross-section coordinate system (the coordinate system representing the position and orientation of a cross section) so that the reference coordinate system is matched with the cross-section coordinate system. Next, the cross-sectional image acquisition unit 130 rotates the cross section in the reference coordinate system so that the orientation of the cross section is matched with the orientation of the ultrasonic tomographic image. Next, the cross-sectional image acquisition unit 130 moves the cross section in parallel so the origin of the cross-section coordinate system is matched with the position of the target lesion area. The cross section calculated in the above manner passes through the target area and is parallel to the ultrasonic tomographic image.

Next, the cross-sectional image acquisition unit 130 calculates a range in which a cross-sectional image is to be generated on the cross section. For example, the range of the image is determined so as to have at least the same size as that of the ultrasonic tomographic image. This is realized by calculating the positions of the four corner points of the ultrasonic tomographic image and generating an area surrounded by the feet of the four perpendiculars extending from the respective four corner points to the cross section as the cross-sectional image.

Finally, the cross-sectional image acquisition unit 130 extracts and generates the image corresponding to the cross section generated in the above manner from the three-dimensional volume data. Since a method of extracting and generating the image corresponding to the cross section that is specified from the three-dimensional volume data is known, a detailed description is omitted herein.

### (S4030) Combination of Images

In Step S4030, the image combining unit 140 combines the ultrasonic tomographic image acquired in Step S4010 with the cross-sectional image generated in Step S4020 to generate a combined image. The display control unit 150 displays the combined image in the display unit 160. The display control unit 150 externally outputs the combined image via the I/F 1009, if needed. In addition, the display control unit 150 stores the combined image in the RAM 1002 so as to allow another application to use the combined image.

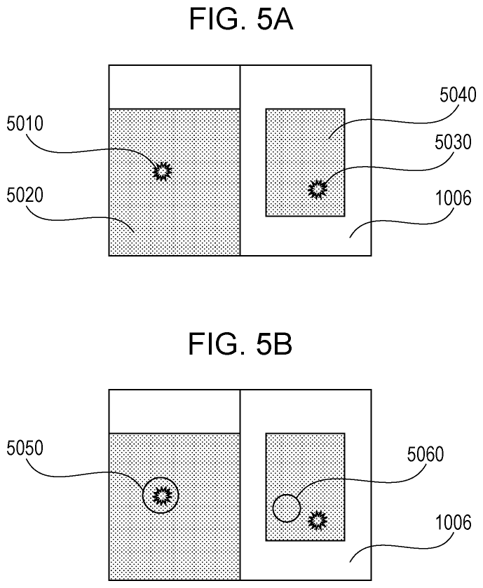

For example, the ultrasonic tomographic image may be drawn in a color different from that of the cross-sectional image and the ultrasonic tomographic image may be superposed on the cross-sectional image for display. Alternatively, only either of the ultrasonic tomographic image and the cross-sectional image may be selectively displayed. Alternatively, the ultrasonic tomographic image may be displayed in one plane resulting from vertical or horizontal division of one screen into two planes and the cross-sectional image may be displayed in the other plane, or the ultrasonic tomographic image and the cross-sectional image are displayed in both of the two planes of the screen. FIG. 5A shows an example in which one screen is vertically divided into two planes and a cross-sectional image 5020 including a target lesion area 5010 and an ultrasonic tomographic image 5040 are horizontally arranged for display. In this example, a corresponding lesion area 5030 is drawn in the ultrasonic tomographic image 5040.

Alternatively, a graphic, such as a circle, indicating the position of the target lesion area may be superposed on the ultrasonic tomographic image for display. This display is realized by drawing a circle resulting from cutting a virtual sphere along the cross section composing the ultrasonic tomographic image on the assumption that the virtual sphere of a certain size is located at the position of the target lesion area. The search for the corresponding lesion area can be based on this graphic in the display. When the position and orientation of the ultrasonic tomographic image is accurately measured and the subject is not deformed, the corresponding lesion area exits at the center of the sphere (that is, the position of the target lesion area). In contrast, if an error in the measurement of the position and orientation of the ultrasonic tomographic image, the difference in posture of the subject at the image capturing, or the deformation of the subject caused by the pressure of the ultrasound probe occurs, the corresponding lesion area does not strictly exist at the center of the sphere. How this problem is resolved will be described in First Modification.

As another example of how to display the specified position, a graphic indicating the position of the target lesion area may be displayed on the cross-sectional image. Alternatively, a graphic, such as an arrow, visually representing the change in orientation of the ultrasonic tomographic image may be displayed. Alternatively, a graphic, such as a plane, representing the position and orientation of the cross-sectional image may be drawn on the three-dimensional volume data that is subjected to volume rendering. Alternatively, whether a graphic is superposed may be selected. FIG. 5B shows an example in which a circle 5050 and a circle 5060 each indicating the position of the target lesion area 5010 are superposed on the cross-sectional image 5020 and the ultrasonic tomographic image 5040, respectively, for display.

Unless a special instruction is input by the operator in Steps S4040 to S4060 described below, Steps S4010 to S4030 are repetitively performed. As a result, the cross-sectional image that includes the target lesion area and that has the same orientation as that of the ultrasonic tomographic image is displayed in the display unit 160 in synchronization with the ultrasonic tomographic image that is sequentially acquired in response to an operation with the ultrasound probe. Accordingly, the operator can easily search for the ultrasonic tomographic image in which the corresponding lesion area is drawn by operating the ultrasound probe while observing the combined image displayed in Step S4030.

The position of the corresponding lesion area in the ultrasonic tomographic image is specified again to correct a shift in position between the target lesion area and the corresponding lesion area in the following Steps S4040 and S4050.

### (S4040) Specification of Position

In Step S4040, the position-orientation acquisition unit 112 determines whether the position of the corresponding lesion area on the ultrasonic tomographic image is specified. The position of the corresponding lesion area is specified, for example, by the operator who clicks a position which the operator considers as the corresponding lesion area on the ultrasonic tomographic image with the mouse 1005. If the position of the corresponding lesion area is specified, the position of the corresponding lesion area in the reference coordinate system is calculated on the basis of the position of the corresponding lesion area and the position and orientation of the ultrasonic tomographic image. Then, the process goes to Step S4050. If the position of the corresponding lesion area is not specified, the process goes to Step S4060.

### (S4050) Correction By Amount of Offset

In Step S4050, the position-orientation acquisition unit 112 calculates the amount of offset between the position of the corresponding lesion area acquired in Step S4040 and the position of the target lesion area acquired in Step S4000. The amount of offset is subtracted from the calculated value of the position of the ultrasonic tomographic image in the subsequent Step S4010 to correct the effect of, for example, the error in the measurement by the position-orientation sensor or the deformation of the subject. Instead of subtracting the amount of offset from the calculated value of the position of the ultrasonic tomographic image, the conversion matrix from the reference coordinate system to the MRI apparatus coordinate system may be varied by the amount of offset. However, the coordinate conversion in Step S4000 is performed again in this case.

### (S4060) Termination

In Step S4060, the information processing apparatus 100 determines whether the overall process is to be terminated. The determination of whether the overall process is to be terminated is input, for example, by the operator who clicks an End button arranged in the display unit 160 with the mouse 1005. If the information processing apparatus 100 determines that the overall process is to be terminated, the overall process in the information processing apparatus 100 is terminated. If the information processing apparatus 100 determines that the overall process is not to be terminated, the process goes back to Step S4010 and Step S4010 to S4060 are performed again to the ultrasonic tomographic image that is newly captured. Here, the operator moves the ultrasound probe in a direction in which the operator considers that the corresponding lesion area is included with reference to the similarity in the shapes of the lesion areas, the appearance of their peripheral parts, or the like.

The cross-sectional image that has the same orientation as that of the ultrasonic tomographic image and that includes the target lesion area is extracted from the three-dimensional image data to generate an image resulting from the combination of the ultrasonic tomographic image and the cross-sectional image in the above manner.

As described above, the cross-sectional image that has the same orientation as that of the acquired ultrasonic tomographic image and that includes the target area (target lesion area) can be extracted from the three-dimensional image data (reference tomographic image group) to display the cross-sectional image. Since the orientation of the extracted cross-sectional image is constantly matched with the orientation of the acquired ultrasonic tomographic image, it is possible to easily search for the corresponding lesion area with reference to the similarity in the shapes of the lesion areas, the appearance of their peripheral parts, or the like.

### First Modification

### Varying Inclination of MRI Cross-Sectional Image Against Deformation

Although the ultrasonic tomographic image has the same inclination with respect to the subject as that of the MRI cross-sectional image that is acquired in accordance with the ultrasonic tomographic image, that is, the ultrasonic tomographic image is parallel to the MRI cross-sectional image in the first embodiment, the exemplary application of the present invention is not limited the above one. It may not be appropriate that the ultrasonic tomographic image is parallel to the MRI cross-sectional image when there is a change in posture of the subject, a deformation of the subject caused by the ultrasound probe, or a variation due to the difference in capturing date. If there is a deformation of the subject caused by the pressing of the ultrasound probe, it is assumed that the subject is deformed because the subject is pressed in the longitudinal direction of the ultrasound probe and the inclination of the MRI cross-sectional image is varied by the amount corresponding to the deformation. The amount of variation may be calculated by using a known deformation model for soft materials.

The generation of the MRI cross-sectional image in consideration of the deformation of the subject allows the MRI cross-sectional image accurately corresponding to the ultrasonic tomographic image to be generated. Accordingly, it is possible to improve the working efficiency of the registration and to realize more accurate registration.

### Second Modification

### Data Other Than MRI Tomographic Image Group

Although the reference tomographic image group is acquired from the data server 190 in the first embodiment, the three-dimensional image data that is used is not limited to the reference tomographic image group. For example, when the data server 190 holds data about an array of luminance values (three-dimensional volume data) that is restored in advance from the reference tomographic image group, the data about the array of luminance values is used as the three-dimensional image data. In this case, the generation of the three-dimensional volume data in Step S4020 may be omitted.

When the data server 190 holds the three-dimensional volume data about the ultrasonic tomographic image, the three-dimensional volume data about the ultrasonic tomographic image is used as the three-dimensional image data. The data server 190 acquires the ultrasonic tomographic images with their position and orientation from the ultrasonic diagnostic imaging apparatus to restore the three-dimensional volume data on the basis of the positional relationship between the tomographic images. In this case, since the tomographic images can be compared with each other on the cross sections of the same orientation even if the ultrasound probe is pressed on the subject in a manner different from that in the past image capturing, the observation of the variation with time of the lesion area can be easily performed.

When the data server 190 holds the three-dimensional volume data that is directly acquired with a three-dimensional ultrasound probe, this three-dimensional volume data may be used as the three-dimensional image data.

### Third Modification

### Tomographic Image Targeted for Registration

The registration may be performed to the tomographic images captured by modalities other than the ultrasonic diagnostic imaging apparatus and the MRI apparatus. In this case, it is possible to easily perform the registration between a first two-dimensional tomographic image captured by a first capturing method and a second two-dimensional tomographic image captured by a second capturing method.

When the tomographic images that are captured are different in the posture of the subject, the image capturing condition, and/or the capturing date and time even if they are captured by the same modality, the tomographic images may be varied. Accordingly, the present invention is applicable to such a case.

### Fourth Modification

### Variation in Display

Although the cross-sectional image of the three-dimensional image data is generated on the basis of the calculated cross section in Step S4020 in the first embodiment, the method of generating the cross-sectional image is not limited to the above one.

For example, the cross-sectional image may be generated by using new three-dimensional volume data whose appearance is adjusted by the image processing. Specifically, the cross-sectional image may be generated from volume data subjected to, for example, edge enhancement or pseudo color processing based on the result of organ segmentation. Alternatively, the cross-sectional image may be generated from volume data subjected to, for example, halftone processing in which the MRI cross-sectional image is converted into an image as if to be captured by the ultrasonic diagnostic imaging apparatus. The cross-sectional image may be subjected to the above image processing after the cross-sectional image is generated from the MRI three-dimensional volume data.

The cross-sectional image to be generated is not limited to the one resulting from imaging of voxel values on a cross section that is calculated as long as the image is generated from the three-dimensional image data on the basis of the calculated cross section. For example, an area that includes the cross section and that has a certain range in the direction of the normal line may be set and a maximum projection image resulting from calculation of the maximum voxel value in the direction of the normal line within the range for each point on the cross section may be used as the cross-sectional image.

### Fifth Modification

### Acquisition of MPR Image With Three-Dimensional Probe

Although the tomographic image is captured by the ultrasonic diagnostic imaging apparatus in the first embodiment, the data acquired by the ultrasonic diagnostic imaging apparatus is not limited to this. For example, the method in the first embodiment is also applicable to a case in which a multi planar reformat (MPR) image is acquired with a three-dimensional ultrasound probe. Specifically, the method in the first embodiment is applied to each of the multiple cross sections.

### Sixth Modification

### Use of Orientation Sensor

The position-orientation sensor is mounted in the ultrasonic diagnostic imaging apparatus to measure the position and orientation of the ultrasound probe in the first embodiment, it is not necessarily measure the position. For example, an orientation sensor may be mounted in the ultrasonic diagnostic imaging apparatus to measure only the orientation of the ultrasound probe.

In this case, the position-orientation acquisition unit 112 calculates the orientation of the ultrasonic tomographic image in the reference coordinate system on the basis of the orientation of the ultrasound probe and the position and orientation of the ultrasonic tomographic image that is calculated in advance.

However, a different method is used to determine the range in which the cross-sectional image is generated by the cross-sectional image acquisition unit 130 in Step S4020 in this case. Specifically, since the positions of the four corner points of the ultrasonic tomographic image are unknown, a certain area around the target area in the cross-sectional image is set as the range where the cross-sectional image is generated. The position of the ultrasonic tomographic image cannot be measured in this case. Accordingly, the drawing of the mark indicating the position of the target lesion area in Step S4030 and the correction of the shift in Steps S4040 and S4050 are not performed.

According to the sixth modification, it is possible to easily find the tomographic image including the corresponding lesion area corresponding to the target lesion area by operating the ultrasound probe with the apparatus of a simpler configuration, compared with the case in which the position-orientation sensor is used.

### Seventh Modification

### Specification of Position of Target Lesion Area

Although the data server 190 holds the position of the target lesion area that is specified in advance in the first embodiment, the position of the target lesion area may be specified in the information processing apparatus. In this case, a lesion specification unit is added to the information processing apparatus.

The lesion specification unit sequentially displays the individual tomographic images composing the three-dimensional image data output from the three-dimensional image data acquisition unit 120 in the display unit 160. The position of the lesion area is specified by the operator who clicks the position on the displayed image, for example, with the mouse 1005 when the target lesion area is displayed in the tomographic image. The position of the target lesion area in the reference coordinate system is calculated on the basis of the position of the lesion area in the tomographic image and the position and orientation of the tomographic image. The above step is performed between Step S4000 and Step S4010.

Alternatively, the information processing apparatus may be configured so that the position of the target lesion area can be reset in the cross-sectional image (for example, the cross-sectional image 5020 in FIG. 5A) displayed in Step S4030. In order to realize the resetting of the position of the target lesion area, a process similar to the acquisition of the position of the corresponding lesion area is also performed to the target lesion area in Step S4040. In this case, the information processing apparatus 100 specifies the position of the target lesion area in response to clicking of the position of the target lesion area in the cross-sectional image displayed in the display unit 160 by the operator with the mouse 1005. The position of the target lesion area is calculated on the basis of the position and orientation of the cross section. With the information processing apparatus according to the seventh modification, it is possible to accurately specify the position of the target lesion area again on the basis of the result of the extraction of the tomographic image including the corresponding lesion area.

### Eighth Modification

### Association and Non-Association

The cross-sectional image having the same orientation as that of the tomographic image is extracted from the three-dimensional image data in the first embodiment. In other words, the method in the first embodiment is effective for the case in which the orientation of the tomographic image is originally matched with the orientation of the cross-sectional image (there is no significant difference in orientation between the tomographic image and the cross-sectional image). However, the present invention is not limited to the above method and the cross-sectional image having an orientation resulting from addition of the amount of offset to the orientation of the tomographic image may be extracted from the three-dimensional image data. For example, a drag operation with the mouse may be performed on the cross-sectional image to set the amount of offset (the rotation axis and the angle of rotation) corresponding to the direction of the drag and the amount of displacement. In this case, the operator feels as if only the cross-sectional image rotates in response to the input. Accordingly, the orientation of the tomographic image can be set in response to the setting of the orientation of the cross-sectional image so that the orientation of the tomographic image is matched with the orientation of the cross-sectional image when they are not matched with each other.

### Ninth Modification

### Method of Setting Orientation

The cross-sectional image having the same orientation as that of the tomographic image is generated in the first embodiment. However, the present invention is not limited to the generation of such a cross-sectional image and a cross-sectional image having an orientation acquired by any method in association with (on the basis of) the orientation of the tomographic image may be generated. For example, the orientation of the cross-sectional image to be generated may be based on the orientation of the past tomographic image (in Step S4020) and the orientation of the current tomographic image. Specifically, the weighted average of the orientation of the past tomographic image and the orientation of the current tomographic image may be set as the orientation of the cross-sectional image. The above method has the advantage of removing a jitter caused by the noise occurring in the measurement of the orientation. Alternatively, the orientation of the cross-sectional image to be generated may be based on the orientation of the cross section at the previous time (in Step S4020) and the orientation of the current tomographic image. Specifically, the weighted average of the orientation of the cross section at the previous time and the orientation of the current tomographic image may be set as the orientation of the cross-sectional image. The above method has the advantage of smoothing effect in which the orientation of the cross section is not sharply varied.

### Second Embodiment

The processing of the ultrasonic tomographic image that is being captured in real time is described in the first embodiment. However, the tomographic image to be processed is not limited to the ultrasonic tomographic image that is being captured in real time and an ultrasonic tomographic image group that is captured in advance may be processed. According to a second embodiment of the present invention, a function is provided to identify the tomographic image including an area corresponding to the target area specified in the three-dimensional image from the ultrasonic tomographic image group that is captured in advance. An information processing apparatus according to the second embodiment will now be described in terms of the difference from the first embodiment.

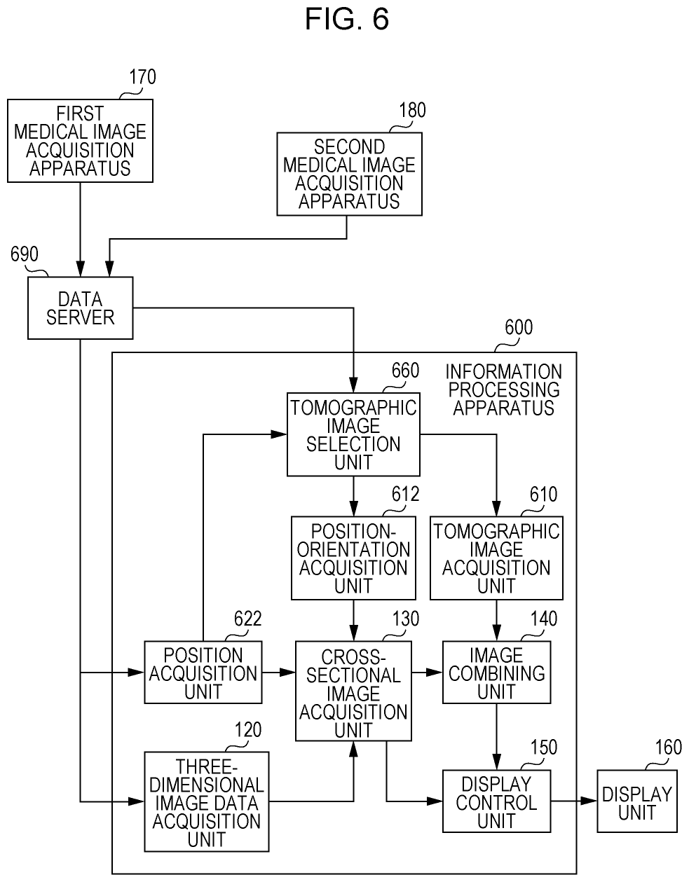

FIG. 6 is a block diagram showing an example of the configuration of an information processing system according to the second embodiment. The same reference numerals and symbols are used in the second embodiment to identify the same blocks in FIG. 1. A description of such blocks is omitted herein. Referring to FIG. 6, an information processing apparatus 600 includes a tomographic image acquisition unit 610, a position-orientation acquisition unit 612, a position acquisition unit 622, and a tomographic image selection unit 660, in addition to the blocks common to those in the first embodiment. The information processing apparatus 600 is connected to a data server 690 holding the three-dimensional image data of the subject that is captured in advance (the same as in the first embodiment) and the ultrasonic tomographic image group.

The ultrasonic tomographic image group held in the data server 690 results from imaging of the subject by the ultrasonic diagnostic imaging apparatus serving as the second medical image acquisition apparatus 180 in advance. The ultrasonic tomographic image group resulting from the imaging of the subject is supplied to the information processing apparatus 600 through the tomographic image selection unit 660. According to the second embodiment, the position and orientation of each ultrasonic tomographic image is also held in the data server 690 and is supplied to the information processing apparatus 600 through the tomographic image selection unit 660.

The position acquisition unit 622 performs the processing in the first embodiment and supplies the position of the target lesion area that is held to the tomographic image selection unit 660. The supply of the position of the target lesion area is performed in response to a request from the tomographic image selection unit 660.

The tomographic image selection unit 660 selects one or more tomographic images from the ultrasonic tomographic image group on the basis of the positional relationship between each ultrasonic tomographic image and the target lesion area. The tomographic image selection unit 660 supplies the selected tomographic image to the tomographic image acquisition unit 610. The tomographic image selection unit 660 supplies the position and orientation of the selected tomographic image to the position-orientation acquisition unit 612.

The tomographic image acquisition unit 610 and the position-orientation acquisition unit 612 differ from the tomographic image acquisition unit 110 and the three-dimensional image data acquisition unit 120 in the first embodiment, respectively, in that the tomographic image acquisition unit 610 and the position-orientation acquisition unit 612 acquire the data output from the tomographic image selection unit 660. Since the tomographic image selection unit 660 outputs the position and orientation of the tomographic image, it is not necessary to calculate the position and orientation of the ultrasonic tomographic image from the position and orientation of the ultrasound probe.

The basic configuration of the computer that realizes the functions of the components composing the information processing apparatus 600 by executing software is the same as in the first embodiment in FIG. 2.

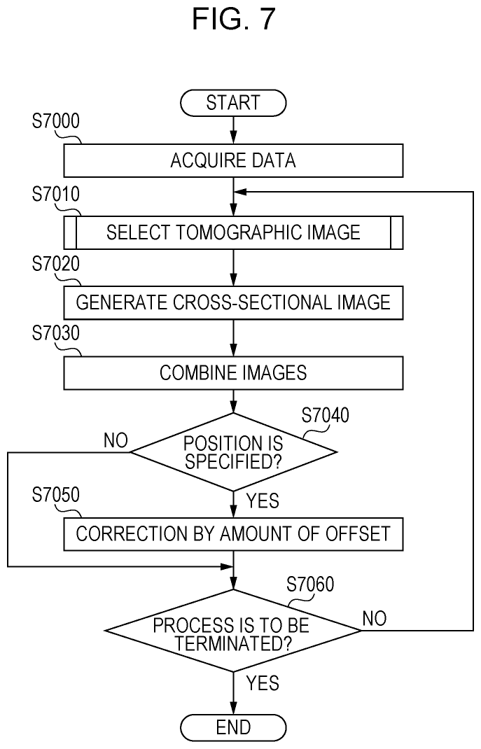

FIG. 7 is a flowchart showing an example of the overall process performed by the information processing apparatus 600. The steps in the flowchart in FIG. 7 are realized by the CPU 1001 that executes the programs realizing the functions of the respective components. It is assumed that, before the following process is started, the program code in accordance with the flowchart has been loaded in the RAM 1002 from, for example, the external storage apparatus 1007.

### (S7000) Acquisition of Data

In Step S7000, the information processing apparatus 600 performs the same processing as in Step S4000 in the first embodiment. In addition, the tomographic image selection unit 660 acquires the ultrasonic tomographic image group and the position and orientation of each ultrasonic tomographic image from the data server 690.

### (S7010) Selection of Tomographic Image

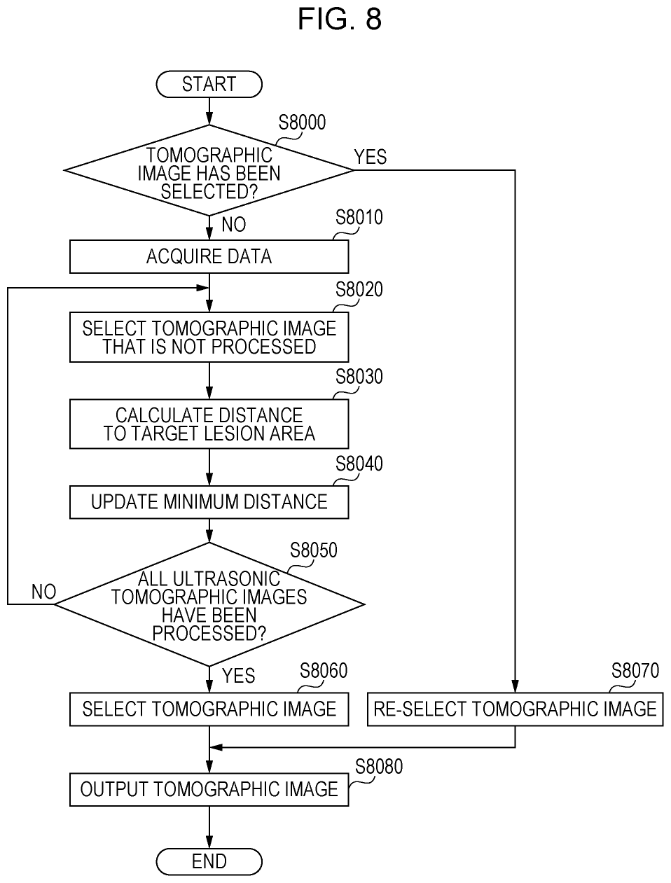

In Step S7010, the tomographic image selection unit 660 selects a selected tomographic image on the basis of the position of the target lesion area and the position and orientation of each ultrasonic tomographic image acquired in Step S7000. The tomographic image selection unit 660 supplies the selected tomographic image to the tomographic image acquisition unit 610 and supplies the position and orientation of the selected tomographic image to the position-orientation acquisition unit 612. The process in the tomographic image selection unit 660 in Step S7010 will be described below in detail with reference to a flowchart in FIG. 8.

Since Steps S7020 to S7060 are similar to Steps S4020 to S4060 in the first embodiment, a detailed description of the steps is omitted herein.

FIG. 8 is a flowchart showing an example of the process in the tomographic image selection unit 660 in Step S7010.

### (S8000) Determination of Selection or Non-Selection

Referring to FIG. 8, in Step S8000, the tomographic image selection unit 660 determines whether the selection of the tomographic image has been performed. If the selection has not been performed, the process goes to Step S8010. If the selection of the tomographic image has been performed, the process goes to Step S8070.

### (S8010) Acquisition of Data

In Step S8010, the tomographic image selection unit 660 acquires the position of the target lesion area from the position acquisition unit 622. In addition, the tomographic image selection unit 660 sets a higher value (for example, 1,000 mm), which is an initial value, as a minimum distance dₘᵢₙ from the ultrasonic tomographic image to the target lesion area.

The tomographic image selection unit 660 selects an ultrasonic tomographic image having the minimum distance to the target lesion area from the ultrasonic tomographic image group in the following Steps S8020 to S8060.

### (S8020) Selection of Tomographic Image That Is Not Processed

In Step S8020, the tomographic image selection unit 660 selects one ultrasonic tomographic image that is not processed from the ultrasonic tomographic image group acquired in Step S7000. For example, the tomographic image selection unit 660 sequentially selects the ultrasonic tomographic images in the order of the capturing time by the ultrasonic diagnostic imaging apparatus.

### (S8030) Calculation of Distance to Lesion Area

In Step S8030, the tomographic image selection unit 660 calculates the distance from the ultrasonic tomographic image selected in Step S8020 to the target lesion area.

Specifically, the tomographic image selection unit 660 calculates the position of the target lesion area in the ultrasonic tomographic image coordinate system of the ultrasonic tomographic image according to Equation (1):

[Math. 1]

xᵢ=x_{w}·T_{iw}⁻¹ (1)

In Equation (1), xᵢ=[xᵢ yᵢ zᵢ 1]^{T} denotes the position of the target lesion area in the ultrasonic tomographic image coordinate system, x_{w}=[x_{w} y_{w} z_{w} 1]^{T} denotes the position of the target lesion area in the reference coordinate system, and T_{iw} denotes a 4 by 4 conversion matrix from the ultrasonic tomographic image coordinate system to the reference coordinate system, representing the position and orientation of the ultrasonic tomographic image.

The tomographic image selection unit 660 calculates a distance d from the ultrasonic tomographic image to the target lesion area according to Equation (2):

d=|zᵢ| (2)

### (S8040) Update of minimum distance

In Step S8040, the tomographic image selection unit 660 determines whether the distance d is smaller than the current dₘᵢₙ. If the distance d is smaller than the minimum distance dₘᵢₙ, the value of the minimum distance dₘᵢₙ is updated to the value of the distance d. The tomographic image selection unit 660 temporarily holds the ultrasonic tomographic image having the minimum distance dₘᵢₙ as the tomographic image closest to the target lesion area.

### (S8050) Determination

In Step 8050, the tomographic image selection unit 660 determines whether all of the ultrasonic tomographic images have been processed. If all the ultrasonic tomographic images have not been processed, the process goes back to Step S8020. If all the ultrasonic tomographic images have been processed, the process goes to Step S8060.

### (S8060) Selection of Tomographic Image

In Step S8060, the tomographic image selection unit 660 selects the ultrasonic tomographic image having the minimum distance to the target lesion area as the selected tomographic image.

When the acquired ultrasonic tomographic image group includes multiple partial tomographic image groups, the tomographic image selection unit 660 performs Steps S8020 to S8060 to each partial tomographic image group. Then, the tomographic image selection unit 660 sequentially selects candidates for the selected tomographic image one by one from each partial tomographic image group and aligns the selected candidates for display in the display unit 160. The tomographic image selection unit 660 selects a final selected tomographic image in response to an instruction from the operator (for example, clicking of a candidate for the selected tomographic image with the mouse).

### (S8070) Re-Selection of Tomographic Image

In Step S8070, the tomographic image selection unit 660 re-selects a tomographic image close to the selected tomographic image. Specifically, the tomographic image selection unit 660 re-selects the tomographic image captured immediately before or after the time when the selected tomographic image is captured as the selected tomographic image. For example, when an instruction “one frame before” is acquired from the operator through an user interface (UI) (not shown), the tomographic image selection unit 660 selects the tomographic image captured one time before the current selected tomographic image as the new selected tomographic image. Similarly, when an instruction “one frame after” is acquired from the operator through the UI, the tomographic image selection unit 660 selects the tomographic image captured one time after the current selected tomographic image as the new selected tomographic image. When an instruction “forward playback” is acquired from the operator, the tomographic image selection unit 660 feeds the tomographic image in the forward direction in accordance with the order of the capturing time each time Step S8070 is performed. In other words, the tomographic image captured at a time just behind is selected. When an instruction “reverse playback” is acquired from the operator, the tomographic image selection unit 660 feeds the tomographic image in the reverse direction in the reverse order of the capturing time each time Step S8070 is performed. In other words, the tomographic image captured at a time just before is selected. When an instruction “stop” is acquired from the operator, the tomographic image selection unit 660 disables the instruction “forward playback” or “reverse playback” (that is, the re-selection of the tomographic image is not performed). The re-selection may be performed in response to any general instruction concerning the display of images in time series. Each of the above instructions may be input, for example, by the operator who selects a specific key to which a command is allocated on the keyboard. Alternatively, an operation button or an operation bar may be arranged on the screen and the instruction may be input by the operator who clicks or drags the operation button or the operation bar with the mouse. When the acquired ultrasonic tomographic image group includes multiple partial tomographic image groups, the partial tomographic image group to be selected is switched in response to an instruction from the operator, as in Step S8060.

When the maximum value of the amount of shift can be estimated from the maximum value of the error in the measurement by the position-orientation sensor or the maximum value of the deformation of the subject, the search range may be restricted. For example, when the maximum value of the amount of shift is equal to 10 mm, only the tomographic image having the distance d within 10 mm, acquired in Step S8030, may be re-selected. Since only the tomographic image in which the corresponding lesion area possibly exists is displayed in this case, it is possible to efficiently perform the search for the corresponding lesion area.

### (S8080) Output of Tomographic Image

In Step S8080, the tomographic image selection unit 660 supplies the selected tomographic image selected in Step S8060 or S8070 to the tomographic image acquisition unit 610. The tomographic image selection unit 660 supplies the orientation of the selected tomographic image to the position-orientation acquisition unit 612. The orientation of the selected tomographic image can be represented by a 3 by 3 rotation matrix R_{iw} composing part of T_{iw}.

As described above, with the information processing apparatus according to the second embodiment, the tomographic image which the operator considers as an image close to the target lesion area is selected from the tomographic image group. Then, the cross-sectional image that includes the target lesion area and that has the same orientation as that of the selected tomographic image can be extracted from the three-dimensional image data (reference tomographic image group). Since the orientation of the extracted cross-sectional image is constantly matched with the orientation of the selected tomographic image, it is possible to easily find the corresponding lesion area with reference to the similarity in the shapes of the lesion areas, the appearance of their peripheral parts, or the like.

### First Modification-1

### Image Other Than Ultrasonic Tomographic Image

The medical image acquisition apparatus that captures the tomographic image is not limited to the ultrasonic diagnostic imaging apparatus. For example, the method in the second embodiment is applicable also when the medical image acquisition apparatus, such as the MRI apparatus or the X-ray CT apparatus, capable of capturing the tomographic image is used.

### Second Modification-1

### Method of Setting Orientation-1

The generation of the cross-sectional image having the same orientation as that of each tomographic image is described in the second embodiment. However, the present invention is not limited to the generation of such a cross-sectional image and a cross-sectional image having an orientation acquired by any method in association with (on the basis of) the orientation of the tomographic image may be generated. For example, the weighted average of the orientation of the tomographic image selected in Step S7010 and the orientation of the tomographic image at a time just before or behind may be set as the orientation of the cross-sectional image to be generated. The above method has the advantage of removing a jitter caused by the noise occurring in the measurement of the orientation. Alternatively, an orientation representative of the partial tomographic image group (representative orientation) may be set as the orientation of the cross-sectional image to be generated. Specifically, the orientation of the tomographic image closest to the target lesion area may be set as the representative orientation or the average of the orientations of the partial tomographic image group may be set as the representative orientation. Since the cross-sectional image of the orientation that is substantially matched with that of the tomographic image is displayed in a still state according to the second modification, the second modification has the advantage of making the image easily viewable because the image is in the still state.

The specification of one tomographic image according to the above embodiments allows the cross-sectional image that is parallel to the tomographic image and that includes the lesion area to be acquired. Consequently, it is not necessary to match the inclinations of the cross sections with respect to the subject with each other and it is sufficient to perform only the registration of the lesion area, thus reducing the workload on the operator.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory devices (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-288454, filed Dec. 18, 2009, which is hereby incorporated by reference herein in its entirety.

1. An information processing apparatus comprising:
a display control unit configured to control display of a cross-sectional image along a first cross section passing through a subject and a cross-sectional image along a second cross section passing through a specified position of the subject;
an acquisition unit configured to acquire an inclination of the first cross section with respect to the subject; and
a setting unit configured to set the second cross section as a cross section that is parallel to the first cross section and that passes through the specified position on the basis of the acquired inclination.

2. (canceled)

3. (canceled)

4. (canceled)

5. (canceled)

6. (canceled)

7. (canceled)

8. (canceled)

9. (canceled)

10. (canceled)

11. (canceled)
